(19) United States
(12) Patent Application Publication
(10) Pub. No.: US 2007/0054397 A1
(43) Pub. Date: Mar. 8, 2007

(54) Adult cardiac uncommitted progenitor cells

(76) Inventors: Harald Ott, Boston, MA (US); Doris Taylor, St. Paul, MN (US)

Correspondence Address: FISH & RICHARDSON P.C., PO BOX 1022, MINNEAPOLIS, MN, 55440-1022, US

(21) Appl. No.: 11/512,009

(22) Filed: Aug. 28, 2006

Related U.S. Application Data
(60) Provisional application No. 60/711,509, filed on Aug. 26, 2005.

(51) Int. Cl. C12N 5/08; C12N 5/06

(52) U.S. Cl. 435/325; 435/366

(57) ABSTRACT

The invention provides for adult cardiac uncommitted progenitor cells (UPCs), methods of making such cells, and methods of using such cells in the treatment, repair and/or regeneration of damaged cardiac tissue.

### CROSS REFERENCE TO RELATED APPLICATIONS

This application claims priority under 35 USC §119(e) to U.S. patent application Ser. No. 60/711,509, filed on Aug. 26, 2005, the entire contents of which are hereby incorporated by reference.

### TECHNICAL FIELD

This invention relates to progenitor cells, and more particularly to adult cardiac uncommitted progenitor cells.

### BACKGROUND

After acute myocardial infarction (MI), damaged cardiomyocytes are gradually replaced by fibrotic, non-contractile tissue. The ventricular dysfunction following MI is primarily due to a massive loss of cardiomyocytes. It is widely accepted that adult cardiomyocytes have little regenerative capability. Therefore, finding new approaches to improve cardiac function after MI or other types of cardiac damage remains a challenge.

### SUMMARY

This disclosure describes a novel population of cardiac-derived progenitor cells that can be isolated from the adult myocardium, expanded, and differentiated into cardiocyte, endothelial and smooth muscle cells.

In one aspect, the invention provides a substantially pure population of adult cardiac uncommitted progenitor cells (UPCs) that express the markers SSEA-1 and/or Oct-4. Such cells can commit to a lineage of mesoderm cells under primary culture conditions. Such mesoderm cells express the marker flk-1 and do not express the marker CD31. Such mesoderm cells express the markers abcg-2, c-kit and sca-1. Such mesoderm cells do not express the markers VECD1, CD45 or SSEA-4. Such mesoderm cells can differentiate into cardiocyte precursor cells (e.g., ventricle cardiocyte precursor cells) expressing the markers nk.x2.5, GATA-4 and/or isl-1 under differentiation culture conditions.

The adult cardiac UPCs described herein are capable of differentiating into endothelial cells, smooth muscle cells, and cardiomyocyte cells. Such endothelial cells express the marker vWF; such smooth muscle cells express the markers SMA and smoothelin; and such cardiomyocyte cells express the markers GATA-4 and MEF-2C. Generally, the SSEA-1 and/or Oct-4 markers are downregulated under differentiation conditions.

In another aspect, the invention provides methods of reducing infarct size in the absence of teratoma production. Such a methods includes the steps of contacting an infarct region with the adult cardiac UPCs described herein. Such adult cardiac UPCs express the markers cardiac MHC and Cn43 following such administration. Such methods also can include monitoring the size of the infarct region before and after the contacting step and also or alternatively can include monitoring ventricular function or performance, fractional shortening (FS), ejection fraction (EF), left ventricular remodeling, left ventricular end-diastolic diameter (LVEDD), and/or left ventricular end-diastolic volume (LVEDV). Such methods generally improves ventricular function or performance, increases fractional shortening (FS), increases the ejection fraction (EF), attenuates left ventricular remodeling, reduces left ventricular end-diastolic diameter (LVEDD), and/or reduces left ventricular end-diastolic volume (LVEDV). In a representative embodiment, the adult cardiac UPCs are autologous to the patient that underwent the infarct. Representative means of contacting include injecting the cells.

In still another aspect, the invention provides methods of repairing or regenerating cardiac tissue in the absence of teratoma production. Such methods include contacting cardiac tissue with the adult cardiac UPCs described herein.

In yet another aspect, the invention provides for methods of making the adult cardiac UPCs described herein. Such methods are disclosed herein and include obtaining adult cardiac tissue, mechanically releasing the adult cardiac UPCs described herein from the heart tissue, and culturing such cells under appropriate conditions for expansion and/or differentiation.

Embryonic cardiac progenitor cells can be identified by cell-surface expression of flk1+, an early marker of lateral mesoderm, in the absence of CD31, an endothelial cell marker. Generally, about 50% to about 80% (e.g., about 55%, 60%, 65%, 70%, or 75%) of the cells in a population of UPCs express flk-1 on the cell surface, while only about 0% to about 10% (e.g., about 2%, 4%, 5%, 6%, or 8%) of the cells exhibit cell-surface expression of CD31. Typically, about 60% to about 100% (e.g., about 65%, 70%, 75%, 80%, 85%, 90%, or 95%) of the cells in such a population express SSEA-1 on the cell-surface. Generally, about 25% to about 50% (e.g., about 30%, 35%, 40%, or 45%) of the cells in a population of UPCs express c-kit on the cell-surface, and about 5% to about 20% (e.g., about 7%, 11%, 14%, 16%, or 19%) of the cells exhibit cell-surface expression of isl-1. Typically, very few cells in a population of UPCs express ABCG2 (e.g., about 0% to about 10%, e.g., about 2%, 4%, 6%, or 8%) or sca-1 (e.g., about 0% to about 10%, e.g., about 2%, 4%, 6%, or 8%) on the surface of the cell.

In one aspect, the invention provides for a substantially pure population of adult cardiac UPCs. Generally, about 50% to about 80% of the cells in a population of adult cardiac UPCs express flk-1 on the cell surface, while only about 0% to about 10% of the cells exhibit cell-surface expression of CD31. Generally, about 60% to about 100% of the cells in such a population express SSEA-1 on the cell surface. Generally, about 25% to about 50% of the cells in a population of adult cardiac UPCs exhibit cell-surface expression of c-kit, and about 5% to about 20% of the cells express isl-1 on the cell surface. Typically, very few cells in a population of adult cardiac UPCs express ABCG2 (e.g., about 0% to about 10%) or sca-1 (e.g., about 0% to about 10%) on the cell surface. A population of adult cardiac UPCs as described herein, when undifferentiated, typically exhibit little to no voltage gated currents. A population of such cells can differentiate into, without limitation, cardiocytes, endothelial cells, and smooth muscle cells.

In another aspect, the invention provides methods of making a substantially pure population of adult cardiac UPCs. Such a method includes providing cells from the ventricles of adult myocardium and expanding such cells on a cardiac-derived mesenchymal feeder monolayer. Under appropriate conditions, the adult cardiac UPCs can differentiate into cardiocytes, endothelial cells, or smooth muscle cells.

It is noted that the adult cardiac UPCs referred to herein are the equivalent or a more purified population of the adult cardiac progenitor cells (ACDPCs) referred to throughout the provisional application to which this application claims benefit.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 shows a schematic depicting the observed recapitulation of cardiac development by UPCs in vitro from an uncommitted SSEA-1+ Oct-4+ state to flk-1+ mesodermal progenitors, to committed endothelial smooth muscle or cardiomyocyte progenitors and finally to differentiating endothelial, smooth muscle and cardiomyocyte cells.

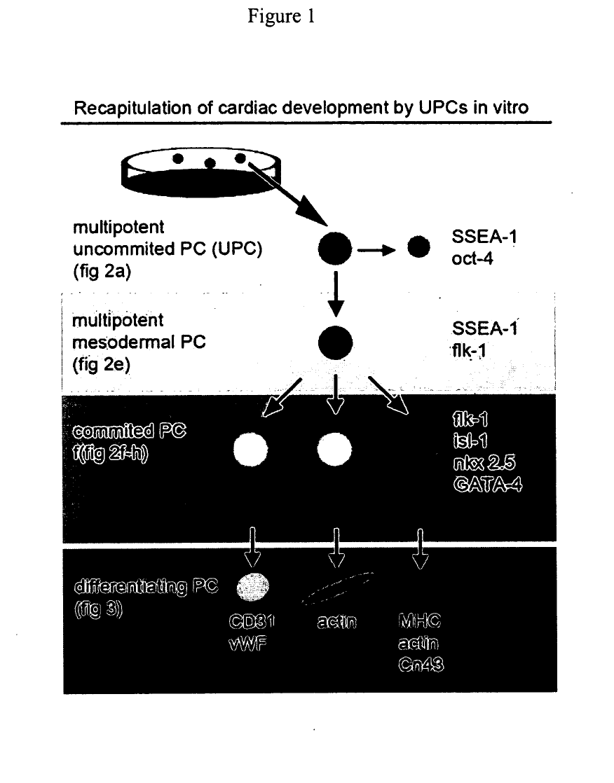

FIG. 2 are graphs that demonstrate functional repair and myocardial regeneration after UPC transplantation in a rat LAD ligation model. Panels A-D show the echocardiographic follow-up at 1-5 weeks following cell treatment. Increased fractional shortening (FS) calculated from M-mode images (Panel A) and ejection fraction (EF) (Panel B) calculated from short and long axis views both show improvement in left ventricular function at 1, 3 and 5 weeks in cell treated animals. Left ventricular end-diastolic diameter (LVEDD) (Panel C) and left ventricular end-diastolic volume (LVEDV) (Panel D) show attenuated left ventricular remodeling after cell transplantation. Panels E-G show invasive hemodynamic measurements at 6 weeks after cell transplantation, which demonstrates improved systolic (dp/dt) (Panel E) and diastolic function (relaxation time tau) (Panel F) and an improved pressure volume relationship (Panel G). All data are shown as mean and standard deviation of the mean, * p<0.05.

FIG. 3 shows the proposed fate of the adult cardiac UPCs disclosed herein—in vitro differentiation and in vivo cardiac repair. (a) Immunohistochemical staining for SSEA-1 (black arrow) in adult left ventricular myocardium. Tissue culture was established from adult myocardial samples. Within 10 days, supernatant SSEA-1+, Oct-4+ uncommitted progenitor cells (UPC) were observed (b). Over time, an increasing number of UPCs maturated into mesodermal progenitor cells expressing flk1 (c). When cultured on a cardiac-derived mesenchymal feeder layer, subpopulations of flk-1+ cells showed co-expression of cardiac stem cell (CSC) markers such as flk-1+, c-kit (d) and sca-1 (f) and the side population cell (SP) marker, abcg-2 (e). Further cardiac commitment was then indicated by expression of the adult cardioblast marker, isl-1(i) and expression of cardiac transcription factors, GATA-4 (g) and nkx2.5 (h). Under endothelial differentiation conditions, flk-1+ cells developed into CD3 1 + endothelial precursor like cells (j). Smooth muscle differentiation conditions induced smooth muscle alpha-actin (SMA) expression and a smooth muscle cell-like phenotype (k). Under appropriate culture conditions, chondrocyte (CHC) (1) and osteoblast-like phenotypes were observed. After injection of GFP-labeled UPCs into infarcted rat myocardium, formation of myosin heavy chain (MHC)-positive grafts and formation of von Willebrand Factor (vWF)-positive cells was observed, indicating in vivo differentiation of UPCs towards cardiomyocyte and endothelium.

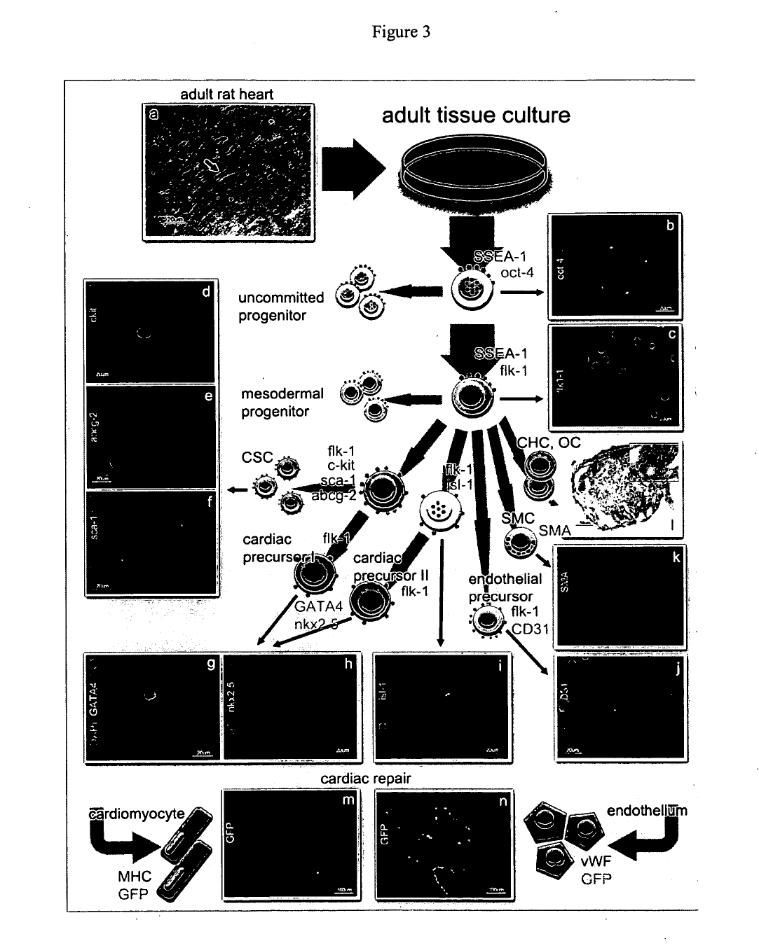

Like reference symbols in the various drawings indicate like elements.

### DETAILED DESCRIPTION

This disclosure describes a novel, substantially pure population of cardiac uncommitted progenitor cells that can be obtained from the adult myocardium, expanded, and differentiated into cardiomyocytes, endothelial cells, and smooth muscle cells. A “substantially pure population” of cells means that at least about 70% (e.g., about 75%, 80%, 85%, 90%, 95%, 99% or 100%) of the cells present (e.g., in the absence of feeder layer cells) are adult cardiac uncommitted progenitor cells (UPCs) as described herein or cells differentiating therefrom. In addition, methods are available for further increasing the number of adult cardiac UPCs in a population. For example, methods such as FACS or methods that use magnetic bead technology can be used to purify one or more subpopulations of adult cardiac UPCs for further expansion.

Adult Cardiac UPCs

The adult cardiac UPCs described herein differ from other previously described adult- or otherwise-derived cardiac progenitor cells. Adult cardiac UPCs in the undifferentiated state express cell surface markers in a pattern similar to embryonic stem cell-derived cardiac progenitor cells (embryonic cardioblasts). Stem cells generally are considered to be undifferentiated when, for example, patch clamp experiments reveal no voltage gated currents in the cells. In addition, transcription factors such as GATA-4 and/or phospholamban (PLM) can be used as markers of undifferentiation.

The adult cardiac UPCs described herein having a SSEA-1+ phenotype can be used to recapitulate the maturation of embryonic stem cells to lateral plate mesoderm and then to cardioblasts. In an undifferentiated and uncommitted state, adult cardiac UPCs express SSEA-1 and/or Oct-4 and do not express isl-1, sca-1, c-kit, nkx2.5, or GATA-4. When the adult cardiac UPCs described herein are removed from the feeder layer, the cells begin differentiating down a committed mesodermal lineage. These committed mesoderm cells express flk-1, abcg-2, c-kit, and sca-1, and do not express CD31, VECD1, CD45, or SSEA-4.

Under differentiation conditions, markers characteristic of stem cells (e.g., SSEA-1 and/or Oct-4) typically are downregulated while cell line specific markers are upregulated. These committed mesodermal cells can further commit to progenitor cells such as smooth muscle progenitor cells that express SMA and smoothelin, cardioblast progenitor cells that express GATA-4, MEF-2C, nkx2.5, and isl-1, or endothelial progenitor cells that express vWF. As used herein, differentiation culture conditions refer to conditions under which the adult cardiac UPCs further differentiate and can be in the presence of, for example, growth factors and/or other differentiated or undifferentiated cell types. In the presence of specific growth factors (e.g., FGF-8b, FGF-4, DKK-1, and BMP-2), the precursor cells can further differentiate. For example, under smooth muscle differentiation conditions, the progenitor cells can differentiate into smooth muscle cells expressing smooth muscle alpha-actin; under cardiac differentiation conditions, the progenitor cells can differentiation into cardiomyocytes expressing cardiac MHC, sarcomeric alpha-actin and connexin43 (Cn43); and under endothelial differentiation conditions, the progenitor cells can differentiation into endothelial cells expressing CD31 and vWF.

In the absence of growth factors (e.g., a feeder layer), adult cardiac UPCs become fully adherent and the expression of SSEA-1, flk1, abcg-2, ckit and sca-1 is downregulated, consistent with the loss of an uncommitted, undifferentiated state. When UPCs remain in primary culture, large (1 mm diameter), adherent, spontaneously beating colonies can form (e.g., on or before about day 14) without additional growth factor stimulation. Beating colonies can be rate-responsive to adrenergic stimulation and stable over 8 weeks in vitro.

It is understood by those of skill in the art that the markers used to describe the phenotype of adult cardiac UPCs generally refer to a protein or a nucleic acid encoding such a protein that can be detected by any number of methods. For example, FACS can be used to detect a cell-surface protein, PCR (e.g., RT-PCR) can be used to detect the presence or absence of a RNA transcript encoding a protein (e.g., a nuclear transcription factor), and immunohistochemistry can be used to detect the presence, absence and/or pattern of distribution of a protein in a cell.

It is also understood by those of skill in the art that the presence or absence of markers can be detected in cells that are grown under a variety of culture conditions. The markers used to describe the adult cardiac UPCs were detected under standard culturing conditions. For example, under the standard culture conditions disclosed below in the Examples, the cells were maintained on a confluent feeder layer at about 1 to 5 million cells. These cells were grown in a standard T75 flask, which holds about 10 to 15 cc of culture media.

Methods of Making and Using Adult Cardiac UPCs

The cells described herein can be obtained by mechanically and enzymatically dissociating cells from the ventricles of mammalian (e.g., rodent or human) myocardial tissue. Mechanical dissociation can be brought about by methods that include, without limitation, chopping and/or mincing the tissue, and/or centrifugation and the like. Enzymatic dissociation from the ECM and/or from cell-to-cell associations can be brought about by enzymes including but not limited to collegenase and trypsin. A population of adult cardiac UPCs can be expanded on a mesenchymal feeder monolayer. A mesenchymal feeder monolayer can be commercially prepared, or can be autologously formed as described herein in the Examples. The adult cardiac UPCs are loosely attached to the monolayer and can be collected in the medium (e.g., in the supernatant). As used herein, “expansion” refers to increasing the number of cells under conditions in which the cells do not undergo a significant amount of differentiation.

The cells described herein also can be obtained and isolated from a patient such that those cells can be transplanted back into the same patient (e.g., autologous transplantation). Such cells can be isolated, for example, by taking an endomyocardial biopsy of the right ventricle or of the interventricular septum via the jugular vein in a standard percutaneous procedure. Using such a method, approximately 1 g of tissue can be obtained from a patient. The tissue can be processed and the cells cultured as described herein.

The cells described herein are capable of myocardial repair and may be superior to skeletal myoblasts when introduced into infarcted or otherwise failing or damaged myocardium. The cells described herein can be used to treat damaged cardiac tissue and also can be used to provide insight into myocardial growth and regeneration. It is understood by those of skill in the art that the terms treating, repairing, replacing, augmenting, improving, rescuing, repopulating or regenerating are used synonymously with respect to disease or damaged tissue.

Introducing cells into cardiac tissue may be accomplished by any means known in the medical arts, including but not limited to grafting and injection. The cells can be introduced into myocardium with or without a natural or artificial support, matrix, or polymer. It should be understood that adult cardiac UPCs can be injected or grafted into or at a site separate and/or apart from the diseased or damaged tissue and allowed to migrate.

As those of skill in the art will understand, a number of factors may be determinative of when and how a stem or progenitor cell differentiates. As a results, it may be desirable to induce differentiation of the cells described herein in a controlled manner and/or by employing factors which are not easily or desirably introduced into the damaged cardiac tissue. Therefore, the cells described herein can be induced to differentiate prior to being introduced into the subject by, for example, in vitro exposure to extracellular and/or intracellular factors such as trophic factors, cytokines, mitogens, hormones, cognate receptors for the foregoing, and the like.

In accordance with the present invention, there may be employed conventional molecular biology, microbiology, cell biology, and biochemical techniques within the skill of the art. Such techniques are explained fully in the literature. The invention will be further described in the following examples, which do not limit the scope of the invention described in the claims.

### EXAMPLES

### Section A. Preliminary Experiments

### Example 1

Isolation of Adult Cardiac UPCs

Six healthy SPF Fisher-344 male rats, aged 8-10 weeks (Harlan Labs; Indianapolis, Ind.), were euthanized by CO₂, briefly submerged in 70% EtOH, and a rapid sternotomy was performed on each using sterile technique. Hearts were excised and placed immediately into 100 mm culture dish containing 12 mL cold 1×PBS (Mediatech; Herndon, Va.). Connective tissue was removed and hearts were transferred to a second 100 mm culture dish containing cold HBSS (Gibco 14025-092) supplemented with 2 mg/mL glucose and 2.5 mg/mL taurine. Ventricular tissue was minced into pieces (<1 mm²) and the sample was pelleted at 25×g for 3 minutes. Samples containing the tissue pellets were digested and sheared with 10 mL of 1 mg/mL collagenase (Gibco 17104-019) and warmed to 37° C. for 15 minutes. The samples then were spun at 25×g for 3 minutes and digested a second time with 10 mL trypsin (0.05%) at 37° C. for 10 minutes. Samples were spun one more time at 25×g for 3 min and the pellet was resuspended in 30 mL CEM at 37° C. T75 flasks (Fisher) were pre-filled with 10 mL CEM (×30) at 37° C. and inoculated with 3 mL of the sample.

Flasks were kept in a 37° C. hydrated cell culture chamber with regulated CO₂ levels of 5-6% (Sanyo Scientific). A visible fibroblast bed developed and tissue chunks became embedded and immobilized. The primary culture flasks were fed every other day with 13 mL of 37° C. CEM. The supernatants from days 2, 4, and 6 were discarded. On day 8, the supernatant from the flasks was collected and spun at 1600 RPM for 14 minutes. Adult cardiac UPCs were used directly, were frozen and used later, or were further enriched as described below.

For further enrichment, pellets were resuspended in 30 mL 37° C. CEM and plated on a fibronectin-coated T75 terminal flask. Terminal flasks were coated with fibronectin by incubating with 5 mg/mL Bovine Plasma Fibronectin (Gibco 33010-018) at 37° C. for 4 hours and washing once with 25° C. PBS. The fibronectin-treated flasks were then placed in the cold room until needed. The fibronectin-treated flasks were inoculated with the day 8 supernatants described above. Two days later (i.e., on day 10), the supernatants were collected and discarded, and 13 mL of CEM were added to the flasks for the initial feeding. The supernatants containing the cells were collected every other day (or more frequently if the cells reached confluency in the terminal flask) and pooled. If desired to increase the yield of cells even further, supernatants from the terminal flasks could be pooled together, and could also be pooled with the supernatants from the primary culture flasks.

Supernatants from the primary culture and/or terminal culture flasks were collected and washed with 1×PBS. Primary culture flasks were exposed to 10 mL 0.025 M EDTA (EM Science OmniPur), and cells were harvested. Flasks were subsequently fed with 13 mL 37° C. CEM and placed into incubators for continued expansion. After collecting the supernatant, some terminal collection flasks were washed once with PBS and trypsinized by an 8-minute exposure to 10 mL of warmed 0.05% Trypsin-EDTA (Gibco 25300-062) to remove and recover cells trapped in the feeder layer. All collections were kept on ice during harvesting.

Cells were spun at 1400 RPM for 12 minutes and the supernatant was gently pulled off using a 2 mL aspirator pipet. Pellets were resuspended in 10 mL PBS and gently washed. A 200 mL sample was taken for viability and quantification analysis using Viacount Assay with Guava machine (Guava technologies, Hayward, Calif.). The final pellet was resuspended in a maximum volume of 320 mL per animal for injection.

### Example 2

Characterization of Adult Cardiac UPCs

The adult cardiac UPCs purified using the above-described methods were transferred and cultured under differentiation conditions (see below). Cells were analyzed using FACS and immunofluorescence before and after differentiation; ion channels through voltage gated channels were recorded using patch clamp technique; and total RNA was isolated from undifferentiated adult cardiac UPCs and RT-PCR analysis was performed for stem cell and cardiac gene expression.

Similar to embryonic cardiac progenitor cells, analysis of the population of undifferentiated adult cardiac UPCs showed a high number of cells expressing flk-1 (60%) and a low number expressing CD31 (5%). In addition, analysis of the population of adult cardiac UPCs showed a high number of cells expressing the embryonic stem cell marker, SSEA-1 (75%). Furthermore, 35% of cells in the population were c-kit⁺ (cardiac stem cell marker), 11% were isl-1⁺ (neonatal cardioblast marker), 6% were sca-1 ⁺ (cardiac progenitor cell marker), and 4% were ABCG2⁺ (cardiac side population marker).

Adult cardiac UPCs that were cultured in endothelial differentiation media exhibited upregulated co-expression of CD31 and flk1 at about day 9. Adult cardiac UPCs cultured in endothelial differentiation media showed spontaneous tube formation at about day 9 and formed a characteristic endothelial cobblestone pattern. Endothelial differentiation media was made as follows: VEGF165 (R&D Systems 293-VE/CF) was reconstituted using PBS/0.1% BSA (Sigma A4503-50G) to a working concentration of 5 mg/mL. The VEGF was added to basal media (see below) to a final concentration of 10 ng/mL. The medium was kept protected from light.

Adult cardiac UPCs that were cultured in smooth muscle differentiation media exhibited upregulated alpha smooth muscle actin expression but downregulated flk1 at about day 9. In smooth muscle differentiation media, the cells morphologically resembled spindle-shaped smooth muscle cells. Smooth muscle differentiation media was made as follows: PDGF-BB (R&D Systems, 220-BB) was reconstituted using 0.1% FBS in 4 mM HCl to a working concentration of 10 mg/mL. 10 mg/mL PDGF was added to basal media (see below) to a final concentration of 5 ng/mL and 10 μg/mL TGF-β1 (R&D Systems) was added to basal media (see below) to a final concentration of 2.5 ng/mL.

Basal media was made as follows: DMEM-LG (Gibco 11885-084) was supplemented with 40% MCDB-201 pH 7.2 (Sigma M-66770), 0.01% ITS liquid media supplement (Sigma 114K8400), 0.01% linoleic acid-albumin (Sigma 094K8407), 0.01% Pen-Strep (10,000 U Pen-G/10,000 mg/mL Strep, Gibco 1237313), 0.01% 10 mM cold L-ascorbic acid (Sigma A8960-5G), 0.0002% 0.25 mM Dexamethasone (Sigma 074K1070), and 0.001% 2-mercaptoethanol (Gibco 21985-023). This media was protected from light and was sterile-filtered using a 0.22 mm filter.

In primary culture on an autologous mesenchymal feeder layer, adult cardiac UPCs grew into spontaneously beating colonies at about day 14.

Preliminary patch clamp experiments showed no voltage gated currents in undifferentiated adult cardiac UPCs.

### Example 3

In Vivo Regenerative Potential

The in vivo regenerative potential of adult cardiac UPCs was tested by injecting the cells into injured myocardium. In a rat coronary artery ligation model of myocardial infarction, 1.2×10⁶ cells of syngeneic adult cardiac UPCs were injected intramyocardially two weeks after injury into the center and border zone of the infarction scar. Myocardial function was assessed using echocardiography and PV loops. Ejection fraction (EF) improved in adult cardiac UPC-treated animals from baseline to week 5 (34.8±4.2 to 56.5±6.5%, p=0.001) but decreased in controls (36.5±3.7 to 28.2±3.8%, p<0.001). At follow up, EF was higher in adult cardiac UPC-treated animals than in controls throughout the 5 weeks (Table 1). LV remodeling was attenuated in adult cardiac UPC-treated animals compared to controls (Table 1). At 6 weeks, maximal +dp/dt was higher and infarct size as determined by morphometry was decreased in adult cardiac UPC-treated animals (Table 1). Relaxation time (τ) was also shorter in adult cardiac UPC-treated animals (Table 1). Based on histology, engraftment of PKH-labeled adult cardiac UPCs within infarction scars was confirmed.

TABLE 1

| | Non-treated | UPC-treated | P value |
|---|---|---|---|
| Baseline: Echo EF | 36.5 ± 3.7 | 34.8 ± 4.2 | p < 0.001 |
| (%) | | | |
| Week 1: Echo EF | 32.8 ± 6.8 | 53.8 ± 10.6 | p < 0.001 |
| (%) | | | |
| Week 3: Echo EF | 28.2 ± 3.3 | 57.7 ± 11.6 | p < 0.001 |
| (%) | | | |
| Week 5: Echo EF | 28.2 ± 3.8 | 56.5 ± 6.5 | p < 0.001 |
| (%) | | | |
| Week 5: LVEDD | 9.1 ± 0.8 | 7.7 ± 0.4 | p < 0.001 |
| (mm) | | | |
| Week 6: Maximal | 3274 ± 962 | 5965 ± 943 | p < 0.001 |
| +dp/dt (mm Hg) | | | |
| Week 6: Relaxation | 22.7 ± 3.7 | 15.8 ± 3.4 | p = 0.008 |
| time (τ) (ms) | | | |
| Infarct size | 25.3 ± 0.16 | 19.6 ± 5.6 | p = 0.043 |
| morphometry (% of | | | |
| LV) | | | |

Adult cardiac UPCs can be isolated from adult ventricular biopsies, expanded in vitro, and differentiated into the major cell types required for myocardial repair. Ejection fraction, +dP/dt and diastolic relaxation each improved significantly after treatment with adult cardiac UPCs when compared to baseline assessment or to vehicle-treated controls. Adult cardiac UPCs engrafted within the infarction scar and reduced infarct size. Adult cardiac UPCs isolated from adult ventricular biopsies improved systolic and diastolic left ventricular function as well as post-infarction remodeling when engrafted within injured myocardium. These results demonstrate that pluripotent cardiac progenitor cells similar to embryonic cardioblasts can be isolated from adult myocardial tissue and effectively used for myocardial repair.

### Section B. Follow-Up Experiments

### Example 1

Tissue Processing and Primary Cell Culture Conditions

Both ventricles of male F344 Fischer rats (Harlan Labs, Indianapolis, Ind.) were excised, dipped into 70% ethanol, and placed into cold phosphate buffered saline (PBS, Mediatech, Hemdon, Va.) supplemented with 2 mg/mL glucose and 2.5 mg/mL taurine (Sigma, St. Louis, Mo.). The tissue was then manually minced into pieces <1 mm³ and centrifuged. The tissue pellet was digested and sheared with in 1 mg/mL Collagenase (Gibco, Carlsbad, Calif.) supplemented with 2 mg/mL glucose at 37° C. for 15 minutes. The mixture was centrifuged again, the supernatant discarded, and the pellet digested with 0.05% Trypsin-EDTA (Gibco, Carlsbad, Calif.) supplemented with 2 mg/mL glucose for 10 minutes. A final centrifugation was performed, and the pellet resuspended in 12 mL basal media (Iscove's Modified Dulbecco's Medium (Gibco, Carlsbad, Calif.), 10% Fetal Bovine Serum (HyClone, Logan, Utah), 100 U/mL penicillin-G (Gibco, Carlsbad, Calif.), 100 U/mL streptomycin (Gibco, Carlsbad, Calif.), 2 mmol/L L-glutamine (Invitrogen, Carlsbad, Calif.), 0.1 mmol/L 2-mercaptoethanol (Gibco, Carlsbad, Calif.)), and plated on uncoated tissue culture flasks. Flasks were kept in humidified (˜60%) cell culture chambers at 37° C. under 5% CO₂ (Sanyo Scientific, Bensenville, Ill.). Primary culture flasks were fed every other day with 12 mL of pre-warmed basal media. Aspirator pipettes were used to remove media as an attached cell layer developed and tissue fragments became embedded and immobilized. Supernatants on days 2, 4, and 6 post-isolation were discarded. On day 8, and every-other-day thereafter, supernatants were collected and spun at 1400 rpm for 14 minutes to isolate suspended cardiac progenitor cells.

For cell transplantation, supernatants of all culture flasks were collected and spun at 1400 rpm for 14 minutes, and the supernatant was removed using a 2 mL aspirator pipette. A 200 μL sample was taken for viability and quantification analysis using Viacount Assay (Guava Technologies, Hayward, Calif.). The final pellet was resuspended in cold PBS to the desired concentration up to a maximum volume of 200 μL per animal for injection. Cells were drawn up into a sterile 1 mL Monoject tuberculin syringe with a 27 gauge needle (Sherwood Medical, St. Louis, Mo.) and kept on ice until needed by the surgical team.

### Example 2

In vitro Differentiation Conditions

Cells were seeded at optimal densities on fibronectin coated (20 ng/mL) or uncoated plastic culture ware. Four-well chamber slides (Lab-Tek, Rochester, N.Y.) or 22×22 mm glass cover slips in 35 mm 6-well plates were fibronectin coated (20 ng/mL, Bovine Plasma Fibronectin, Gibco, Carlsbad, Calif.) according to the manufacturers instructions. Cells were plated at low density for smooth muscle (˜3000 cells/cm²) and medium density for cardiomyocyte, endothelial and osteocyte differentiation (˜45,000 cells/cm²). Fractional media changes of 50% were made every two days to induce cardiomyocyte, endothelial and smooth muscle differentiation. Basal differentiation media (DMEM-LG (Gibco, Carlsbad, Calif.), 40% MCDB-201 (Sigma, St. Louis, Mo.), 0.01% ITS liquid media supplement (Sigma), 0.01% Linoleic acid-albumin (Sigma, St. Louis, Mo.), 0.01% Pen-Strep (Gibco, Carlsbad, Calif.), 0.1% 10 mM cold L-Ascorbic acid (Sigma, St. Louis, Mo.), 0.0002% 0.25mM Dexamethasone (Sigma, St. Louis, Mo.), 0.001% 2-Mercaptoethanol (Gibco, Carlsbad, Calif.)) was supplemented with different growth factors depending on the target cell type. For example, for endothelial cell differentiation, 10 ng/ml VEGF165 (R&D Systems, Minneapolis, Minn.) was added; for smooth muscle differentiation, 5 ng/ml PDGF-BB (R&D Systems, Minneapolis, Minn.) and 2.5 ng/ml human TGF-β1 (R&D Systems, Minneapolis, Minn.) were added; for cardiomyocyte differentiation, 10 ng/ml recombinant mouse FGF-8b (R&D Systems, Minneapolis, Minn.), 100 ng/ml FGF-4 (R&D Systems, Minneapolis, Minn.), 10 ng/ml recombinant Human Dkk-1 (R&D Systems, Minneapolis, Minn.), 10 ng/ml recombinant human Bone Morphogenetic Protein 2 (R&D Systems, Minneapolis, Minn.) and 0.75% Dimethyl Sulphoxide (Sigma, St. Louis, Mo.) were added; for osteocyte differentiation, 0.1 mM Dexamethasone (Sigma, St. Louis, Mo.), 10 mM Glycerophosphate disodium salt hydrate (Sigma, St. Louis, Mo.) and 0.2 mM L-Ascorbic Acid (Sigma, St. Louis, Mo.) were added; and for chondrocyte differentiation, 5 ng/mL BMP-4 (R&D Systems, Minneapolis, Minn.) was added.

### Example 3

GFP Transfection and Co-culture Conditions

Cells were transfected using Green Fluorescent Protein Adenovirus (Ad-GFP)—Type 5 with CMV promoter (Vector Biolabs, Philadelphia, Pa.). The virus was administered with the third feeding on Day 6 post-tissue processing at 1×10⁹ IFU/mL media.

Neonatal cardiomyocytes were processed using a commercially available isolation system following the manufacturers specifications (Worthington Biochemical Corp., Freehold, N.J.). For indirect co-culture, neonatal cells were plated in 2.0 mLs Neonatal-Cardiomyocyte Explant Media at densities of 125,000 cells/cm² on fibronectin-coated (20 ng/mL) 6-well co-culture plates (Coming Transwell, 3450—Clear, 24 mm Diameter, 0.4 um pore size, Coming, N.Y.). Into the co-culture mesh-basket, ACBs were plated at a density of 50,000 cells/cm² in 1.0 mL cardiomyocyte differentiation media. The ACB-containing mesh-basket was then secured into its housing, submerging approximately 1 mm into the media. There was no physical contact between these two cell populations in coculture.

### Example 4

Reverse Transcription and Real-Time Quantitative RT-PCR

One microgram of total RNA was reverse-transcribed using random hexamers and Moloney murine leukemia virus (MMLV) reverse transcriptase (Life Technologies, Gaithersburg, Md.). Oligonucleotide primers were designed according the corresponding human cDNA sequences in the National Institutes of Health GenBank. mRNA levels were determined by quantitative real-time RT-PCR analysis, using the Light Cycler Thermocycler (Roche Diagnostics, Basel, Switzerland). Reactions were prepared in the presence of the fluorescent dye, SYBR green I, for specific detection of double-stranded DNA. Quantification was performed in the log-linear phase of the reaction, and cycle numbers obtained at this point were plotted against a standard curve prepared from serially diluted control samples. Results were normalized to GAPDH expression levels.

### Example 5

Animal Heart Failure Model and Cell Transplantation

Myocardial infarction was induced by LAD ligation in eight week old F344 Fischer rats as previously described (Ott et al., 2004, Eur. J. Cardiothorac. Surg., 25:627-34). The adult derived cardioblasts described herein were isolated and expanded from ventricular myocardium of three F344 rats (12 weeks of age). Cell characteristics were confirmed by FACS analysis and immunofluorescence. Two weeks after infarction, baseline echocardiographies were performed and animals were divided into cell treated and control group (animals with an ejection fraction >40% were excluded). Either 200 μl of saline containing 1×10⁶ cells (UPC group, n=11) or 200 μl of saline (control group, n=13) were directly injected into six sites in and around the infarction scar as previously described (Ott et al., supra). For functional follow-up, transthoracic echocardiography was performed one, three and five weeks after treatment. Pressure volume loops were recorded six weeks after injection and hearts were processed for histological analysis.

### Example 6

Echocardiography and Invasive Hemodynamic Measurements

Left ventricular function was assessed by two-dimensional echocardiography recorded on a Sonos 5500 (Philips, Andover, Mass.) using a 15 MHz linear array transducer two weeks after infarction and one, three and five weeks after cell transplantation or saline injection. Under general anesthesia with 1.5% Isoflurane, the chest was shaved and a layer of acoustic coupling gel was applied. M-mode, parastemal long and short axis views were recorded and analyzed by two blinded readers. Fractional shortening (FS) and left ventricular end diastolic diameter (LVEDD) were measured on M-mode images. Ejection fraction (EF) and left ventricular end diastolic volume (LVEDV) were calculated using a modified Simpson formula from end systolic and end diastolic short and long axis areas assuming an ellipsoid shape of the left ventricle.

Invasive hemodynamic measurements were performed at 6 weeks after cell transplantation or saline injection. Under general anesthesia (Ketamine at 10 mg/100 g and Xylazine at 1 mg/100 g administered intraperitoneally), a 2 cm incision was performed in the right neck. The right common carotid artery was dissected and a pressure catheter (Millar Instruments, Houston, Tex.) was inserted. The catheter was advanced into the left ventricle and pressure was recorded after an initial stabilization period using Sonolab for MSDOS (Sonometrics, London, Ontario, Canada). After recording was completed, the catheter was pulled back into the ascending aorta to minimize hemodynamic compromise.

A median sternotomy was performed in the lower two thirds of the sternum, the heart was carefully exposed and four sonomicrometry crystals (1 mm, Sonometrics, London, Ontario, Canada) were sutured to the epicardium of the anterior and posterior left ventricular wall, the apex and the base of the left ventricle using 7/0 Prolene sutures (Sherwood Medical, St. Louis, Mo.). The pressure catheter was again advanced into the left ventricle and sonometric measurements were recorded using Sonolab for MSDOS (Sonometric, London, Ontario, Canada). After obtaining a baseline recording, the right inferior caval vein was occluded using a micro bulldog clamp to record left ventricular pressure and sonometry under a different preload. After completion of these measurements, the crystals were carefully removed and the heart was harvested for histological analysis.

### Example 7

Histology and Morphometry

Immunofluorescence staining was performed on primary cultures, freshly isolated cells from suspension, differentiated progenitor cells and cell treated hearts. Chamber slides were spun at 500 rpm for 10 min to attach suspended cells to the surface of the slide. Supernatant was aspirated and harvested cultured cells were fixed with 4% Paraformaldehyde (Electron Microscopy Sciences, Hatfield, Pa.) in 1×PBS (Mediatech, Herndon, Va.) for 15 mins at room temperature. The fixed cells were permeabilized with 0.1% Triton X 100 (Sigma, St. Louis, Mo.) for 10 min at room temperature. Fixed cells were blocked with 4% Fetal Bovine Serum (HyClone, Logan, Utah) in 1×PBS for 30 minutes at room temperature. Samples were sequentially incubated for one hour at room temperature with diluted primary and secondary antibodies (Ab). Between each step, slides were washed 3 times (5-10 min each) with 1×PBS. Primary antibody (Ab) against Flk-1 (rabbit polyclonal IgG, Cat# sc-315, Santa Cruz Biotechnology Inc., Santa Cruz, Calif.) and Ab against CD-31 (goat polyclonal IgG, Cat# sc-1506, Santa Cruz Biotechnology Inc.) were used at a 1:40 dilution with blocking buffer. Secondary Ab's bovine anti-rabbit IgG phycoerythin (Santa Cruz Biotechnology Inc. Cat# sc-3750) and bovine anti-goat IgG FitC (Santa Cruz Biotechnology Inc. Cat# sc-2348) were used at a 1:80 dilution with blocking buffer. Slides were covered with cover glass (Fisherbrand 22×60) in hardening mounting medium containing 4′,6-Diamidino-2-phenylindole (DAPI) (Vectashield, Vector Laboratories, Inc., Burlingame, Calif.). Images were recorded using ImagePro Plus 4.5.1 (Mediacybemetics, Silver Spring, Md.) on a Nikon Eclipse TE200 inverted microscope (Fryer Co. Inc., Huntley, Ill.).

For morphometry, 24 sections were uniformly sampled from one series of Massons's-Trichrome stained sections for each rat heart and photographed using a Nikon Eclipse TE200 microscope. The areas of scar and left ventricle (LV) were quantified with the color segmentation-based method in ImagePro Plus 4.5.1 (Mediacybernetics, Silver Spring, Md.). The interventricular septum was considered together with the LV. The relative volume percentage of scar was computed as [p=sum(scar area)/sum(left ventricular area)]. One-side compared t-test was performed for the two groups (cell treated and sham).

### Example 8

Data Analysis

Functional data were analyzed using SPSS 11.0 for Windows (SPSS Inc., Chicago, Ill.). Data are expressed as mean±standard deviation. Comparisons of continuous variables among animal groups and longitudinal studies comparing data within each group were achieved by the use of paired t-tests. A value of p<0.05 was considered significant.

### Example 9

Characterization of Uncommitted Progenitor Cells

With increasing time in primary culture, UPCs showed a shift in surface marker expression from an SSEA-1+ to a flk1+ phenotype, consistent with a maturation of the suspended cell population from an immature, uncommitted SSEA-1+ state to a mesoderm committed flk1+ state. Among the supernatant cells, small populations of abcg-2+, ckit+ and sca-1+ cells also were observed. All abcg2+ and sca-1+ cells and 50% of the c-kit+ cells coexpressed flk1. No expression of CD31 (PECAM-1), VE-cadherin (VECD1), CD45 or SSEA-4 was observed among supernatant SSEA-1+ and flk1+ cells, confirming their undifferentiated state.

A population of uncommitted progenitor cells was identified that express the embryonic stem cell marker stage specific embryonic antigen-1 (SSEA-1) throughout both neonatal and adult (weeks 14 and 24, respectively) rat ventricular myocardium. In the neonatal heart, SSEA-1+ cells were dispersed among mature cardiomyocytes throughout the heart, whereas in the adult heart, SSEA-1+ cells remained as single cells in both right and left ventricle and as clusters in the right ventricular outflow tract. In the neonatal but not in the adult heart, the SSEA-1+ cells did express the early cardiomyocyte markers nkx2.5, GATA4 and cardiac myosin heavy chain (MHC), indicating their likely role as cardiocyte precursors. In adult myocardium, only SSEA-1 was expressed, suggesting only uncommitted progenitors persist; consistent with an uncommitted phenotype, the SSEA-1+ cells did not express isl-1, sca-1 or c-kit. In the present study, the SSEA-1+ progenitor cell population was isolated and expanded in an uncommitted, undifferentiated state from adult rat myocardium, in vitro differentiation was performed into multiple lineages, and the cardiac repair potential of the cells was tested in a rat heart failure model.

To isolate UPCs, ventricular myocardium from adult F344 Fischer rats was enzymatically digested and used to generate a cardiac mesenchymal feeder layer above which immature SSEA-1+ UPCs expanded in single cell suspension. One gram of myocardium led to a cumulative cell yield of 4.5×10⁸ cells over ˜90 days of culture—sufficient cell numbers for clinical application in patients post ischemic injury. In vitro, UPCs expressed SSEA-1 and Oct-4 comparable to undifferentiated mouse embryonic stem cells.

Over time, subpopulations of supernatant flk1+ cells further committed to a cardiac lineage as evidenced by expression of nkx2.5, GATA4, and the recently defined RV cardioblast marker isl-1. The number of these cardiac progenitor cells increased with the age of primary cultures (nkx2.5+ 5%, GATA4 4% and isl1+ 4% at week 2; Nkx2.5+ 16%, GATA4 12% and isl1+ 11% at week 5). This further maturation of the supernatant progenitor cell population from an uncommitted SSEA-1+ through a mesoderm committed flk1+/CD31− progenitor cell to a cardiac committed precursor cell parallels the maturation from embryonic stem cells to lateral plate mesoderm and then to cardioblasts. Importantly, UPCs gave rise to two distinct populations of cardiac precursor cells: nkx2.5/GATA-4+ cells and isl-1+ cells. In cardiac development, isl1+ expression is required for the development of atria, right ventricle and outflow tract, whereas nkx2.5 and GATA4 expression is a prerequisite for the development of the left ventricular field.

As with a developing heart and embryonic stem cells, for example, UPCs could not mature in isolation without specific growth factor stimulation. Thus, when UPCs were expanded without a feeder layer or in the absence of defined growth factors, they became fully adherent and underwent downregulation of SSEA-1, flk1, abcg-2, ckit and sca-1. This change is consistent with the loss of the uncommitted, undifferentiated state of murine and human embryonic stem cells when they are removed from a mesenchymal feeder layer and cultured under non-specific conditions.

When UPCs remained in primary culture, large (1 mm diameter), adherent, spontaneously beating colonies formed by day 14 without additional growth factor stimulation. These beating colonies were rate-responsive to adrenergic stimulation and stable over 8 weeks in vitro. To promote cardiomyocyte differentiation under specified in vitro conditions, purified UPCs were cultured without a feeder layer but in the presence of FGF-8b, FGF-4, DKK-1, and BMP-2. Beginning at day 4, multicellular clusters were observed that were positive for sarcomeric α-actin, cardiac MHC, and connexin43 (Cn43). This characteristic also is similar to the results obtained with flk-1+ ESCs, in which inhibition of the wnt/β-catenin pathway via DKK1 induces cardiac differentiation. Although growth factor stimulation induced up-regulation of cardiac transcription factors (see below) and synthesis of characteristic cardiac structural proteins, spontaneous contraction was not observed.

To promote further differentiation, adenovirus GFP-labeled UPCs were directly co-cultured with neonatal rat cardiomyocytes. GFP+ cells expressed cardiac MHC and formed spontaneously beating colonies starting at co-culture day 3. To evaluate the importance of cell-cell contact, UPCs were seeded onto a transwell mesh (0.8 μm pore size) that was co-cultured but not in direct contact with rat neonatal cardiomyocytes. Even though there was no direct cell-to-cell contact between neonatal cardiomyocytes and UPCs, spontaneously beating colonies formed at day 4.

Under smooth muscle differentiation conditions (e.g., in the presence of PDGF-BB and TGF-β1), UPCs elongated and expressed smooth muscle α-actin. Endothelial cell culture conditions (e.g., in the presence of VEGF-165) induced an endothelial phenotype and expression of CD31. Osteocyte differentiation (e.g., in the presence of Glycerol 2-Phosphate Disodium Salt Hydrate) induced calcium deposition. Chondrocyte differentiation (e.g., in the presence of BMP-4 in suspension culture) induced formation of a chondrocyte pellet.

To further characterize primary cultures of UPCs (supernatant UPCs plus adherent feeder layer), isolated UPCs (suspended cells only) and differentiating UPCs, quantitative RT-PCR was performed for various stem cell and differentiation markers. In primary heart derived culture and UPCs, expression of SSEA-1, oct-4, c-kit and sca-1 was confirmed. SSEA-1 and oct-4 expression in UPCs was comparable to undifferentiated mouse embryonic stem cells (mESC). In UPCs isolated at different time points, expression of SSEA-1, ckit and sca-1 remained constant, while oct-4 increased to a maximum at week 6 and then declined, which is consistent with changing cell commitment of supernatant UPCs, as precise levels of oct-4 expression determine commitment to germ layers or self-renewal. During endothelial, cardiac and smooth muscle differentiation, a downregulation of SSEA-1, oct-4, c-kit and sca-1 was observed starting at day 3.

Together with a downregulation of stem cell markers, upregulation of cell line specific genes was observed in UPCs exposed to differentiation conditions. Primary cultures at day 14 (supernatant UPCs plus feeder layer) showed a baseline expression of endothelial, cardiac and smooth muscle markers, indicating the presence of cells at differing stages of commitment to these cell lineages. Isolated UPCs (supernatant UPCs only) showed lower levels of these markers, confirming their immature and undifferentiated state.

Endothelial differentiation conditions induced upregulation of vWF. Cardiac differentiation conditions lead to upregulation of the cardiac transcription factors, GATA-4 and MEF-2C, consistent with cardiac lineage commitment. Smooth muscle differentiation conditions led to upregulation of the early smooth muscle marker, SMA, and the later marker, smoothelin, indicative of a pattern of UPC smooth muscle differentiation observed in embryonic smooth muscle development.

### Example 10

Role of UPCs in Cardiac Repair

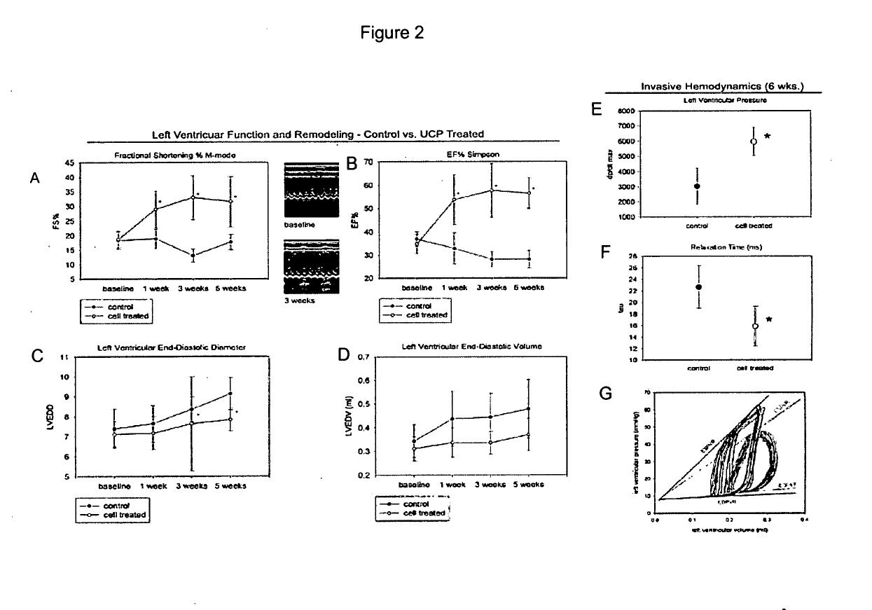

To test the potential of UPCs to mediate cardiac repair, intramyocardial injection of isogeneic adult heart-derived UPCs was performed two weeks after myocardial infarction in rats. Cell-treated hearts showed smaller infarct size than control hearts (0.196±0.057% vs. 0.253±0.016%, p=0.044). Injected UPCs formed large isolated grafts within scar tissue, similar to the engraftment pattern observed after transplantation of immature neonatal cardiomyocytes, but no evidence of teratoma was seen, which was in direct contrast to the result obtained after injection of undifferentiated mESCs. Engrafted GFP+ UPCs expressed cardiac MHC and Cn43, indicating differentiation to a cardiomyocyte phenotype. UPCs were oriented with the cardiac fiber direction, however, showed an immature phenotype similar to neonatal cardiomyocytes. GFP+ UPCs expressed vWF, indicating their contribution to angiogenesis and differentiation into endothelial cells. Left ventricular function improved in UPC-treated animals from baseline to week 5, but decreased in controls (FIG. 2A,B). Fractional shortening (FS) and ejection fraction (EF) were higher in UPC-treated animals than in controls throughout the 5 weeks (FS: p=0.798 baseline, p<0.001 week 1 and 3, p=0.005 week 5; EF: p=0.218 baseline, p<0.001 week 1, 3 and 5). Left ventricular remodeling was attenuated in UPC-treated animals (FIG. 2C,D). Left ventricular end-diastolic diameter (LVEDD) was smaller in UPC-treated animals than in controls at week 3 and 5 (LVEDD: p=0.394 baseline, p=0.230 week 1, p=0.026 week 3, p=0.001 week 5). Accordingly, left ventricular end-diastolic volume (LVEDV) was reduced in cell-treated animals (LVEDV: p=0.273 baseline, p=0.020 week 1, p=0.006 week 3, p=0.020 week 5).

At week 6 after cell transplantation, invasive hemodynamic measurements confirmed improved left ventricular performance as a higher maximal +dp/dt in UPC-treated animals compared to controls (p<0.001) (FIG. 2E). Relaxation time (tau) was decreased in UPC-treated animals, indicating improved diastolic function (p=0.008) (FIG. 2F). In P/V recordings, UPC-treated animals showed a higher end-systolic elastance than control animals, which supports the observed positive effect of cell treatment (FIG. 2G).

In summary, SSEA-1+, Oct-4+ UPCs can be derived from adult rat myocardium, isolated and expanded in vitro to yield a large number of cells. These precursors can be induced to differentiate down endothelial, smooth muscle and cardiocytes lineages under controlled in vitro conditions, suggesting their potential for cardiac repair and regeneration. After ischemic injury, UPCs can mediate cardiac functional repair by all criteria examined, and contribute to the formation of vascular and cardiomyocyte-like cells. This novel population of adult heart derived cells can provide new options for autologous adult cardiac repair.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially pure population of adult cardiac uncommitted progenitor cells (UPCs), wherein said adult cardiac UPCs express the markers SSEA-1 and/or Oct-4.

2. The adult cardiac UPCs of claim 1, wherein said cells commit to a lineage of mesoderm cells under primary culture conditions.

3. The adult cardiac UPCs of claim 2, wherein said mesoderm cells express the marker flk-1 and do not express the marker CD31.

4. The adult cardiac UPCs of claim 3, wherein said mesoderm cells express the markers abcg-2, c-kit and sca-1.

5. The adult cardiac UPCs of claim 3, wherein said mesoderm cells do not express the markers VECD 1, CD45 or SSEA-4.

6. The adult cardiac UPCs of claim 2, wherein said mesoderm cells differentiate into cardiocyte precursor cells expressing the markers nk.x2.5, GATA-4 and/or isl-1 under differentiation culture conditions.

7. The adult cardiac UPCs of claim 6, wherein said cardiocyte precursor cells are ventricle cardiocyte precursor cells.

8. The adult cardiac UPCs of claim 1, wherein said UPCs are capable of differentiating into endothelial cells, smooth muscle cells, and cardiomyocyte cells.

9. The adult cardiac UPCs of claim 8, wherein said endothelial cells express the marker vWF.

10. The adult cardiac UPCs of claim 8, wherein said smooth muscle cells express the markers SMA and smoothelin.

11. The adult cardiac UPCs of claim 8, wherein said cardiomyocyte cells express the markers GATA-4 and MEF-2C.

12. The adult cardiac UPCs of claim 1, wherein the SSEA-1 and/or Oct-4 markers are downregulated under differentiation conditions.

13. A method of reducing infarct size in the absence of teratoma production, comprising the steps of:
contacting an infarct region with the adult cardiac UPCs of claim 1.

14. The method of claim 13, wherein said adult cardiac UPCs express the markers cardiac MHC and Cn43 following said administration.

15. The method of claim 13, further comprising monitoring the size of the infarct region before and after said contacting step.

16. The method of claim 13, further comprising monitoring ventricular function or performance, fractional shortening (FS), ejection fraction (EF), left ventricular remodeling, left ventricular end-diastolic diameter (LVEDD), and/or left ventricular end-diastolic volume (LVEDV).

17. The method of claim 13, wherein said method improves ventricular function or performance, increases fractional shortening (FS), increases the ejection fraction (EF), attenuates left ventricular remodeling, reduces left ventricular end-diastolic diameter (LVEDD), and/or reduces left ventricular end-diastolic volume (LVEDV).

18. The method of claim 13, wherein said adult cardiac UPCs are autologous to the patient that underwent said infarct.

19. The method of claim 13, wherein said contacting comprises injecting said cells.

20. A method of repairing or regenerating cardiac tissue in the absence of teratoma production, comprising the steps of:
contacting cardiac tissue with the adult cardiac UPCs of claim 1.
